(19) United States
(12) Patent Application Publication
(10) Pub. No.: US 2016/0213575 A1
(43) Pub. Date: Jul. 28, 2016

(54) COATING COMPOSITIONS, DENTAL STRUCTURES THEREOF AND METHODS FOR GENERATING CONTRAST

(71) Applicant: 3M INNOVATIVE PROPERTIES COMPANY, Saint Paul, MN (US)

(72) Inventors: Yizhong Wang, Woodbury, MN (US); Joel D. Oxman, Minneapolis, MN (US); Bradley D. Craig, Lake Elmo, MN (US)

(21) Appl. No.: 14/917,216

(22) PCT Filed: Sep. 3, 2014
(86) PCT No.: PCT/US14/53814

§ 371 Date: Mar. 7, 2016

Related U.S. Application Data
(60) Provisional application No. 61/876,432, filed on Sep. 11, 2013.
(60) Provisional application No. 61/876,448, filed on Sep. 11, 2013.

(51) Int. Cl. A61K 6/00; A61M 31/00; A61C 17/00; A61C 9/00; C09D 133/08; C09D 133/02

(52) U.S. Cl. CPC A61K 6/002; C09D 133/08; C09D 133/02; A61K 6/0094; A61C 17/00; A61C 9/004; A61M 31/005; C08K 3/22

(57) ABSTRACT

A coating composition and a dental structure are described. The coating composition can include an acidic copolymer having acidic acrylate monomeric units, acidic methacrylate monomeric units, or a combination thereof; a solvent having ethanol and water; and a pigment. The coating composition can comprise two phases and the wt- % of each component is based on the total weight of the composition. The dental structure can include a dental article, and a film on a surface of the dental article, wherein the film is formed by drying the coating composition on the dental article. A method for generating contrast for oral scanning on a dental structure and a method for scanning a dental structure are described.

### CROSS REFERENCE TO RELATED APPLICATIONS

This application claims the benefit of U.S. Provisional Application Nos. 61/876432 and 61/876448 filed Sep. 11, 2013, which are incorporated herein by reference in their entirety.

### FIELD

The present disclosure generally relates to coating compositions, e.g. oral coating compositions and dental structure thereof, and methods for generating contrast, e.g. methods for generating contrast for oral scanning on a dental structure.

### BACKGROUND

Some existing dental 3D scanner can capture dental tissues and dimensions in short amount of time, but such scanner required additional contrast on the surface of the dental tissues to get fast and accurate scanning. Current dental scanning technology used a titanium dioxide powder dusted on dental tissues to create contrast.

### SUMMARY

Titanium dioxide used in some existing dental scanning technologies is difficult to handle and needs an expensive and specially designed delivery device to dust dental tissues. Such titanium dioxide powder does not have sufficient adhesion to dental structures and can be easily moved around by saliva, tongue and the scanner wand. The resulted change of titanium dioxide powder pattern renders the scanning difficult. In addition, the titanium dioxide powder can cause discomfort (e.g., a rough intraoral surface sensation).

The present disclosure generally relates to oral coating compositions and dental structure thereof. Generally, the oral coating composition of the present disclosure can create a contrast coating with durability during scanning and with easy removal property after scanning.

Some aspects of the present disclosure provide a coating composition. The coating composition can include an acidic copolymer comprising acidic acrylate monomeric units, acidic methacrylate monomeric units, or a combination thereof, wherein the coating composition comprises no greater than 10 wt- % of the acidic copolymer; a solvent comprising ethanol and water, wherein the coating composition comprises from about 35 to about 45 wt- % of ethanol and from about 45 to about 60 wt- % of water; and from about 0.05 to about 10.0 wt- % of a pigment. The coating composition can comprise two phases and the wt- % of each component is based on the total weight of the composition.

Some aspects of the present disclosure provide a dental structure. The dental structure can include a dental article, and a film on a surface of the dental article, wherein the film is formed by drying the above coating composition on the dental article.

Some aspects of the present disclosure provide a method for generating contrast for oral scanning on a dental structure. The method can include providing an oral coating composition, and coating a dental structure with the oral coating composition. The oral coating composition can include an acidic copolymer comprising acidic acrylate monomeric units, acidic methacrylate monomeric units, or a combination thereof; a solvent comprising ethanol and water; and a pigment. The oral coating composition can have two phases.

Some aspects of the present disclosure provide a method for scanning a dental structure. The method can include providing an oral coating composition, coating a dental structure with the oral coating composition; and scanning the dental structure with the oral coating composition.

Other features and aspects of the present disclosure will become apparent by consideration of the detailed description and accompanying drawings.

### BRIEF DESCRIPTION OF THE DRAWINGS

FIG. 1 is a captured scan of bovine tooth with coating Ex 3.

FIG. 2 is a captured scan of tooth model with coating Ex 3.

FIG. 3 is a schematic side view of a dental structure according to an embodiment of the invention.

### DETAILED DESCRIPTION

Before any embodiments of the present disclosure are explained in detail, it is understood that the invention is not limited in its application to the details of use, construction, and the arrangement of components set forth in the following description or illustrated in the following drawings. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

The present disclosure generally relates to coating compositions, e.g. oral coating compositions and dental structure thereof. Particularly, for example, the coating compositions of the present disclosure can provide a discontinuous coating to generate texture, pattern or different contrast on the surface of the dental structures so that the dental scanner can capture images of the dental structures.

As used herein, dental structures include, but are not limited to, dental tissues and dental articles.

As used herein, dental tissues include, but are not limited to hard and soft dental tissues. Hard and soft oral tissues include, but not limited to, teeth, dental arch, and the surrounding tissues and support structures including gingiva and hard palate.

As used herein, dental articles include, but are not limited to an article that can be attached (e.g., bonded) to dental tissues (e.g., a tooth structure). Examples of dental articles include, but are not limited to, replacements, inlays, onlays, veneers, full and partial crowns, bridges, implants, implant abutments, copings, dentures, posts, bridge frameworks and other bridge structures, abutments, orthodontic appliances and devices including, but not limited to archwires, buccal tubes, brackets and bands, and prostheses (e.g., partial or full dentures).

As used herein, an aqueous solution includes, but is not limited to water, saliva, artificial saliva or combinations thereof.

In some embodiments, a coating composition is provided. The coating composition of the present disclosure can include an acidic copolymer, a solvent and a pigment. The acidic copolymer can have acidic acrylate monomeric units, acidic methacrylate monomeric units, or a combination thereof. The solvent can include ethanol and water. In some embodiments, the coating composition can comprise from about 0.05 to about 10.0 wt- % of the pigment. In some embodiments, the coating composition can comprise from about 35 to about 45 wt- % of ethanol. In some embodiments, the coating composition can comprise from about 45 to about 60 wt- % of water. In some embodiments, the coating composition comprises no greater than 10 wt- % of the acidic copolymer. The composition can include two phases and the wt- % of each component is based on the total weight of the composition.

Acidic copolymers can, for example, lower the pH of the oral composition. As a result, acidic copolymers can be used as film formers. In some embodiments, the acidic copolymer can include, but is not limited to, an acidic acrylic copolymer of a monomeric unit selected from the group consisting of acrylic acid, methacrylic acid and combinations thereof.

In some embodiments, the acidic copolymer can include a copolymer of methacrylic acid and methyl methacrylate. In some other embodiments, the acidic copolymer can include Eudragit® S100 (marketed by Evonic Industries AG, Damstadt, Germany), Eudragit® L100 (marketed by Evonic Industries AG, Damstadt, Germany), Eudragit® L100-55 (marketed by Evonic Industries AG, Damstadt, Germany), AC210 (marketed by The Lubrizol Corporation, Wickliffe, Ohio, USA), or combinations thereof.

In some embodiments, the molecular weight of the acidic copolymer can be from about 5,000 to about 500,000. In other embodiments, the molecular weight of the neutral copolymer can be from about 10,000 to about 100,000.

In some embodiments, the neutral acrylate monomeric units can include, but are not limited to, isobutyl acrylate, tert-butyl acrylate, 2-ethylhexyl acrylate, lauryl acrylate, lauryl/tridecyl acrylate, cetyl acrylate, stearyl acrylate, cyclohexyl acrylate, benzyl acrylate, isobornyl acrylate, 2-methoxyethyl acrylate, 2-ethoxyethyl acrylate, 2-ethoxyethoxyethyl, acrylate, 2-phenoxyethyl acrylate, tetrahydrofurfuryl acrylate, 2-hydroxyethyl acrylate, 2-hydroxypropyl acrylate, 4-hydroxybutyl acrylate, 1,4-butanediol acrylate, and combinations thereof. In some other embodiments, diacrylates can include the diacrylates of: 1,4-butanediol, 1,6-hexanediol, tetraethylene glycol, tripropylene glycol, and ethoxylated bisphenol-A. In other embodiments, triacrylate monomers include those of: trimethylol propane, ethoxylated, glyceryl propoxy, and pentaerythritol.

In some embodiments, neutral methacrylate monomeric units can include, but are not limited to, methyl methacrylate, ethyl methacrylate, n-butyl methacrylate, isobutyl methacrylate, tert-butyl methacrylate, 2-ethylhexyl methacrylate, lauryl methacrylate, alkyl methacrylate, tridecyl methacrylate, stearyl methacrylate, cyclohexyl methacrylate, benzyl methacrylate, isobornyl methacrylate, 2-hydroxyethyl methacrylate, 2-hydroxypropyl methacrylate, dimethylaminoethyl methacrylate, diethylaminoethyl methacrylate, glycidyl methacrylate, tetrahydrofurfuryl methacrylate, allyl methacrylate, ethylene glycol methacrylate, triethylene glycol methacrylate, tetraethylene glycol methacrylate, 1,3-butyleneglycol methacrylate, 1,6-hexanediol methacrylate, trimethylopropane methacrylate, ethoxyethyl methacrylate and trifiuoroethyl methacrylate, and combinations thereof.

In order to be useful as a scanning composition, the oral composition described herein comprises a pigment that can be detected by the imaging device. In the case of optical scanning techniques, the oral composition typically comprises a pigment that creates a micron-scale roughness contributing to diffuse, Lambertian surface reflection characteristics. Hence, such agent can be described as a diffusely reflecting particulate agent.

Different pigments can be dispersed in the coating to indicate the location and help the oral scanner to capture images. These pigments can be organic or inorganic pigments. Organic pigments can include, but are not limited to, cadmium orange and chrome orange. Inorganic pigments can include, but are not limited to, aluminum pigments, iron pigments, copper pigments, titanium pigments, tin pigments, clay earth pigments, carbon pigments, barium pigments, zinc pigments with many different colors and their combinations. In some embodiments, the pigment can be titanium dioxide.

Although (e.g. food grade) TiO₂, has been commonly described as a component of surface treatment compositions for intra-oral scanning, various other pigments can be utilized provided that the material of the pigment sufficiently differs in refractive index from the composition. Suitable pigments can include, but are not limited to, ZnO, ZnO₂, BaSO₄, talc, as well as various particulate food materials such as rice powder.

In some embodiments, the coating composition is capable of forming a film on a surface, when contacted with an aqueous solution or by drying the coating composition. In some other embodiments, the coating composition is capable of forming a discontinuous film on a surface, when contacted with an aqueous solution or by drying the coating composition. The discontinuous film is formed, for example, due to phase separation of the copolymer. The two phases of the coating composition can include a copolymer rich phase and a solvent rich phase. Such a discontinuous film, for example, can generate a texture, a pattern or a different contrast on dental tissue surface, including without limitation tooth surface to obtain fast and accurate scanning. As a result, such discontinuous film, for example, can provide a higher contrast pattern to ease the scanning. In some other embodiments, the coating composition is capable of forming the film in less than about 30 seconds, when contacted with an aqueous solution or by drying the coating composition with a stream of compressed air.

In some embodiments, the coating composition can be suitable for scanning a dental structure.

Various methods can be employed to apply the coating composition on the dental structure. In some embodiments, the coating composition can be applied from the composition's container or dispenser such as a bottle, syringe, or tube. In some embodiments, a dental brush, microfiber, foam or sponge applicator or cotton Q tip is used to rub the surface of the dental structure and leave a thin layer of coating on the surface. In some other embodiments, a tray applicator, a dental tray, or a dental strip filled with the coating composition can be used. The coating composition can cover the surface of the dental structure and leave a layer of coating on the surface. In other embodiments, the coating composition can be sprayed (e.g. air-brushing) with a spray device or aerosol applicator onto the surface of the dental structure. In other embodiments, the coating composition can be directly painted onto the surface of the dental structure with a brush tip attached to a syringe. In yet other embodiments, the coating composition can be applied as a rinse. The coating composition can be set into a coating on the dental structure and its attachments within 30 seconds by water, saliva, or dried by air blowing.

The coating composition, for example, can be used as an adhesive to provide a good adhesion to the surface. The discontinuous film formed by the coating composition is durable and thus the pattern of the discontinuous film is substantially maintained during scanning. The discontinuous film, for example, can resist the disturbance by saliva during scanning, the contact with a scanning wand, or brushing, so that the discontinuous film can stay sufficient long on the surface. Therefore, the oral composition of the present disclosure can provide prolonged coating/film. In some embodiments, the film remains on at least 90% of the surface after brushing the surface for at least 5 strokes. The brushing stroke is defined as brushing the surface back and forth one time. In some other embodiments, the film remains on at least 90% of the surface after brushing the surface for at least 10 strokes. In other embodiments, the film remains on at least 90% of the surface after brushing the surface for at least 20 strokes. In yet other embodiments, the film remains on at least 90% of the surface after brushing the surface for at least 30 strokes. In some cases, the film remains on at least 90% of the surface after brushing the surface for at least 60 strokes. In other cases, the film remains on at least 90% of the surface after brushing the surface for at least 90 strokes. In yet other cases, the film remains on at least 90% of the surface after brushing the surface for at least 120 strokes.

In some embodiments, the film remains on at least 90% of the surface after brushing the surface for from 5 to 120 strokes. In some other embodiments, the film remains on at least 90% of the surface after brushing the surface for from 10 to 90 strokes. In other embodiments, the film remains on at least 90% of the surface after brushing the surface for from 20 to 60 strokes. In yet other embodiments, the film remains on at least 90% of the surface after brushing the surface for from 5 to 120 strokes.

The film formed by the coating composition can be removed after applied with an alkaline solution. Acidic copolymer of the present disclosure can be dissolved in the alkaline solution and thus can be brushed away from the dental tissues with the alkaline solution after scanning.

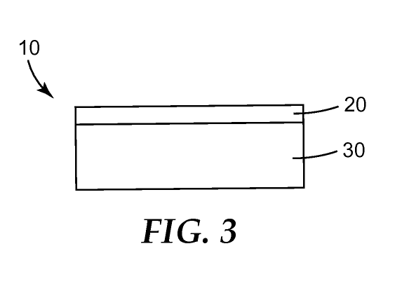

As shown in FIG. 3, in some embodiments, a dental structure 10 is provided. The dental structure 10 can include a dental article 30 and a film 20 on a surface of the dental article. The film can be formed by drying the above-described coating composition on the dental article.

In some embodiments, the dental article can be chosen from teeth, crowns, bridges, inlays, onlays, veneers, facings, copings, crown and bridged framework, implants, abutments, and orthodontic appliances. In other embodiments, the dental article is a monolithic crown. In some implementations, the dental article is chosen from brackets, buccal tubes, cleats and buttons.

In some embodiments, a method for generating contrast for oral scanning on a dental structure is provided. The method can include providing an oral coating composition described herein, and coating a dental structure with the oral coating composition. The oral coating composition can include an acidic copolymer, a solvent and a pigment. The acidic copolymer can have acidic acrylate monomeric units, acidic methacrylate monomeric units, or a combination thereof. The solvent can include ethanol and water. The oral coating composition can have two phases.

In some embodiments, a method for scanning a dental structure is provided. The method can include providing an oral coating composition described herein, coating a dental structure with the oral coating composition and scanning the dental structure with the oral coating composition. The oral coating composition can include an acidic copolymer, a solvent and a pigment. The acidic copolymer can have acidic acrylate monomeric units, acidic methacrylate monomeric units, or a combination thereof. The solvent can include ethanol and water. The oral coating composition can have two phases.

In some other embodiments, the method of scanning the dental structure can further include removing the coating composition from the dental structure after the scanning. In other embodiments, removing the coating composition from the dental structure after the scanning can include applying an alkaline solution to the dental structure. In yet other embodiments, removing the coating composition from the dental structure after the scanning can further include brushing the dental structure after applying the alkaline solution to the dental structure. Acidic copolymer of the present disclosure can be dissolved in the alkaline solution and thus can be brushed away from the dental tissues with the alkaline solution after scanning. One exemplary advantage of applying the alkaline solution to the dental structure is to provide an easy removal of the coating composition. Such easy removal, for example, can be due to the reaction of the acidic copolymer with the alkaline solution.

The following embodiments are intended to be illustrative of the present disclosure and not limiting.

### EMBODIMENTS

Embodiment 1 is a coating composition, comprising

an acidic copolymer comprising acidic acrylate monomeric units, acidic methacrylate monomeric units, or a combination thereof, wherein the coating composition comprises no greater than 10 wt- % of the acidic copolymer;

a solvent comprising ethanol and water, wherein the coating composition comprises from about 35 to about 45 wt- % of ethanol and from about 45 to about 60 wt- % of water; and

from about 0.05 to about 10.0 wt- % of a pigment;

wherein the coating composition comprises two phases; and

wherein the wt- % of each component is based on the total weight of the composition.

Embodiment 2 is the coating composition of embodiment 1, wherein the coating composition is capable of forming a film on a surface, when contacted with an aqueous solution or by drying the oral composition.
Embodiment 3 is the coating composition of any preceding embodiment, wherein the coating composition is capable of forming a discontinuous film on a surface when contacted with an aqueous solution or by drying the oral composition.
Embodiment 4 is the coating composition of any preceding embodiment, wherein the coating composition is suitable for scanning a dental article.
Embodiment 5 is the coating composition of any preceding embodiment, wherein the coating composition is capable of forming the film by drying the coating composition.
Embodiment 6 is a dental structure, comprising:

a dental article, and

a film on a surface of the dental article, wherein the film is formed by drying the coating composition on the dental article;

wherein the coating composition comprises:

an acidic copolymer comprising acidic acrylate monomeric units, acidic methacrylate monomeric units, or a combination thereof, wherein the composition comprises no greater than 10 wt- % of the acidic;

a solvent comprising ethanol and water, wherein the coating composition comprises from about 35 to about 45 wt- % of ethanol and wherein from about 45 to about 60 wt- % of water; and

from about 0.05 to about 10.0 wt- % of a pigment;

wherein the coating composition comprises two phases; and

wherein the wt- % of each component is based on the total weight of the composition.

Embodiment 7 is the dental structure of embodiment 6, wherein the dental article is chosen from teeth, crowns, bridges, inlays, onlays, veneers, facings, copings, crown and bridged framework, implants, abutments, and orthodontic appliances.
Embodiment 8 is the dental structure of embodiments 6-7, wherein the dental article is a monolithic crown.
Embodiment 9 is the dental structure of embodiment 6-8, wherein the dental article is chosen from brackets, buccal tubes, cleats and buttons.
Embodiment 10 is the coating composition or the dental structure of any preceding embodiment, wherein the molecular weight of the acidic copolymer is from about 5,000 to about 500,000.
Embodiment 11 is the coating composition or the dental structure of any preceding embodiment, wherein the acidic copolymer is chosen from Eudragit S100, Eudragit L100, Eudragit L100-55, AC210 and combinations thereof.
Embodiment 12 is the coating composition or the dental structure of any preceding embodiment, wherein the pigment is chosen from cadmium orange, chrome orange, aluminum pigments, iron pigments, copper pigments, titanium pigments, tin pigments, clay earth pigments, carbon pigments, barium pigments, zinc pigments and combinations thereof.
Embodiment 13 is the coating composition or the dental structure of any preceding embodiment, wherein the pigment is chosen from TiO₂, ZnO, ZnO₂, BaSO₄, talc, rice powder and combinations thereof.
Embodiment 14 is the coating composition or the dental structure of any preceding embodiment, wherein the pigment is TiO₂.
Embodiment 15 is a method for generating contrast for oral scar on a dental structure comprising:

providing an oral coating composition, and

coating a dental structure with the oral coating composition;

wherein the oral coating composition comprises:
an acidic copolymer comprising acidic acrylate monomeric units, acidic methacrylate monomeric units, or a combination thereof;
a solvent comprising ethanol and water; and
a pigment; and

wherein the oral coating composition comprises two phases.

Embodiment 16 is a method for scanning a dental structure comprising:

providing an oral coating composition,

coating a dental structure with the oral coating composition; and

scanning the dental structure with the oral coating composition

wherein the oral coating composition comprises:
an acidic copolymer comprising acidic acrylate monomeric units, acidic methacrylate monomeric units, or a combination thereof;
a solvent comprising ethanol and water; and
a pigment; and

wherein the oral coating composition comprises two phases.

Embodiment 17 is the method of claim 16, further comprising removing the coating composition from the dental structure after scanning the dental structure with the oral coating composition.
Embodiment 18 is the method of claim 17, wherein removing the coating composition from the dental structure after the scanning comprises applying an alkaline solution to the dental structure.
Embodiment 19 is the method of claim 18, wherein removing the coating composition from the dental structure after the scanning further comprises brushing the dental structure after applying the alkaline solution to the dental structure.

The following working examples are intended to be illustrative of the present disclosure and not limiting.

### EXAMPLES

Objects and advantages of this invention are further illustrated by the following examples, but the particular materials and amounts thereof recited in these examples, as well as other conditions and details, should not be construed to unduly limit this invention.

The materials used to prepare examples of the invention (Ex) as well as comparative examples (CE) are outlined below.

### Materials

| Material | Description | Source |
|---|---|---|
| EUDRAGIT | Acidic anionic copolymer | Evonic Industries, |
| L100-55 | based on methacrylic acid | Darmstadt, Germany |
| | and ethyl acrylate | |
| EtOH | Ethanol, 200 proof, USP | Columbus Chemical |
| | grade | Industries, Columbus, WI |
| Micro TiO₂ | Titanium dioxide colorant | Evonic Degussa Corp, |
| | | Pasippany, NJ |
| Precolor TiO₂ | Titanium dioxide colorant | Universal Preserv-a- |
| | | chem, Somerset, NJ |

### Comparative Example 1 (CE 1)

A polymer solution was made by mixing 45 g ethanol, 45 g water and 10 g EUDRAGIT L100-50 in a 250 ml glass jar which was then sealed with a cap. The sealed jar was then placed on a Wheaton Culture Roller and rolled for 1 day at 60 rpm to form a clear solution. Using a small dental adhesive brush the coating composition was applied to tooth brushed bovine teeth and then dried using a blow drier to form a continuous film coating on the teeth. The teeth were scanned using a 3M True Definition Scanner (available from 3M Company, St Paul, Minn.). The scanner failed to capture the tooth structure when the uniform coating was used.

### Examples 1-5 (Ex 1-5) and Comparative Examples 2-3 (CE 2-3)

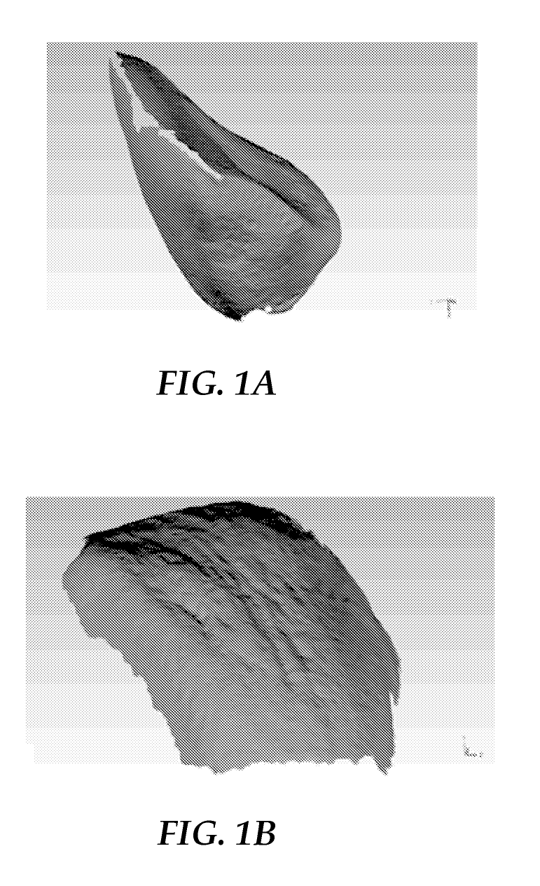

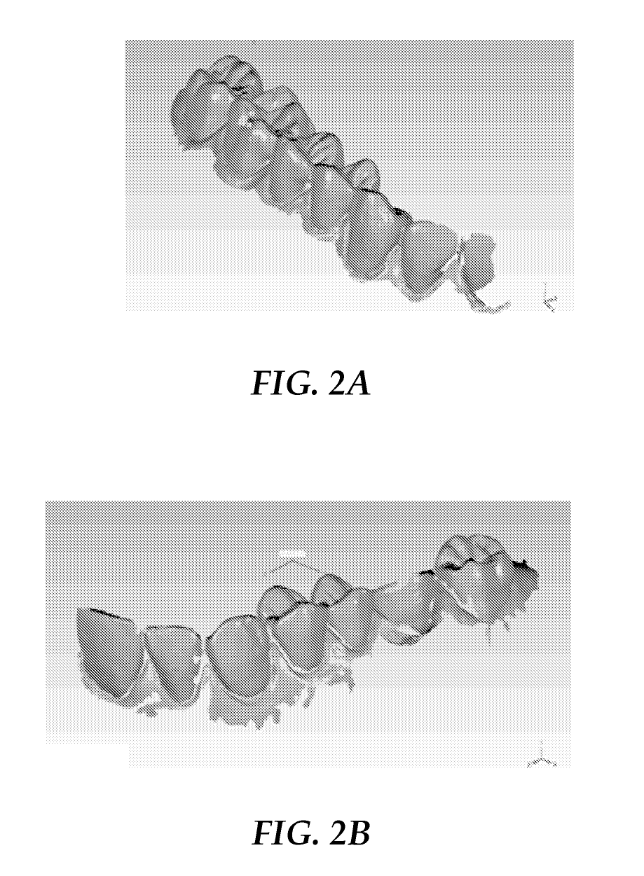

Two phase polymer dispersions were prepared by mixing the amount of polymer, ethanol and water designated in Table 1 in a 250 ml glass jar which was then sealed with a cap. The sealed jar was then placed on a Wheaton Culture Roller and rolled for 1 day at 60 rpm. The resulting polymer dispersion was then heated to 50° C. for 1 hour and formed a clear solution which upon cooling formed cloudy dispersion. To this dispersion, TiO₂ was added as outlined in Table 2 using a speed mixer at 3000 rpm for 2 minutes. The resulting dispersion was then coated onto cleaned bovine teeth or tooth models using a small dental adhesive brush. The thin coating layer was then dried using a blow dryer or by dipping the coated substrate in water to set the liquid coating into a non uniform coating. The coated teeth or models were scanned using a 3M True Definition Scanner and the scanning contrast (scan quality) was evaluated. The results are shown in Table 2. Actual scans of the bovine teeth and tooth models coated with the composition from Ex 3 are shown in FIGS. 1 and 2 respectively.

TABLE 1
Polymer Dispersions (PD)

| | PD1 | PD2 | PD3 |
|---|---|---|---|
| EUDRAGIT L100-50 | 10 | 5 | 2 |
| Ethanol | 40 | 42 | 43 |
| Deionized water | 50 | 53 | 55 |
| Character at room | Two phase | Two phase | Two phase |
| temperature | dispersion | dispersion | dispersion |

TABLE 2
Contrast Coatings

| | Ex 1 | Ex 2 | Ex 3 | Ex 4 | Ex 5 | CE 1 | CE 2 |
|---|---|---|---|---|---|---|---|
| PD1 | 20 | 0 | 0 | 0 | 0 | 0 | 0 |
| PD2 | 0 | 20 | 0 | 0 | 0 | 0 | 0 |
| PD3 | 0 | 0 | 20 | 20 | 20 | 20 | 20 |
| Micro TiO₂ | 0 | 0 | 0 | 0 | 0.05 | 0 | 0.020 |
| Precolor TiO₂ | 0.2 | 0.2 | 0.2 | 0.05 | 0 | 0.020 | 0 |
| Scanning Contrast | good | good | good | good | good | difficult | difficult |

After scanning, the contrast coating on the teeth and tooth model was easily removed by wetting the coating with a 10% aqueous NaCO₃ solution and then brushing away the dissolved coating with a tooth brush.

1. A coating composition, comprising:
an acidic copolymer comprising acidic acrylate monomeric units, acidic methacrylate monomeric units, or a combination thereof, wherein the coating composition comprises no greater than 10 wt- % of the acidic copolymer;
a solvent comprising ethanol and water, wherein the coating composition comprises from about 35 to about 45 wt- % of ethanol and from about 45 to about 60 wt- % of water; and
from about 0.05 to about 10.0 wt- % of a pigment;
wherein the coating composition comprises two phases; and
wherein the wt- % of each component is based on the total weight of the composition.

2. The coating composition of claim 1, wherein the coating composition is capable of forming a film on a surface, when contacted with an aqueous solution or by drying the oral composition.

3. The coating composition of claim 1, wherein the coating composition is capable of forming a discontinuous film on a surface when contacted with an aqueous solution or by drying the oral composition.

4. The coating composition of claim 1, wherein the coating composition is suitable for scanning a dental article.

5. The coating composition of claim 1, wherein the coating composition is capable of forming the film by drying the coating composition.

6. A dental structure, comprising:
a dental article, and
a film on a surface of the dental article, wherein the film is formed by drying the coating composition on the dental article;
wherein the coating composition comprises:
an acidic copolymer comprising acidic acrylate monomeric units, acidic methacrylate monomeric units, or a combination thereof, wherein the composition comprises no greater than 10 wt- % of the acidic;
a solvent comprising ethanol and water, wherein the coating composition comprises from about 35 to about 45 wt- % of ethanol and wherein from about 45 to about 60 wt- % of water; and
from about 0.05 to about 10.0 wt- % of a pigment;
wherein the coating composition comprises two phases; and
wherein the wt- % of each component is based on the total weight of the composition.

7. The dental structure of claim 6, wherein the dental article is chosen from teeth, crowns, bridges, inlays, onlays, veneers, facings, copings, crown and bridged framework, implants, abutments, and orthodontic appliances.

8. The dental structure of claim 6, wherein the dental article is a monolithic crown.

9. The dental structure of claim 6, wherein the dental article is chosen from brackets, buccal tubes, cleats and buttons.

10. The dental structure of claim 6, wherein the molecular weight of the acidic copolymer is from about 5,000 to about 500,000.

11. The dental structure of claim 6, wherein the acidic copolymer is chosen from Eudragit S100, Eudragit L100, Eudragit L100-55, AC210 and combinations thereof.

12. The dental structure of claim 6, wherein the pigment is chosen from cadmium orange, chrome orange, aluminum pigments, iron pigments, copper pigments, titanium pigments, tin pigments, clay earth pigments, carbon pigments, barium pigments, zinc pigments and combinations thereof.

13. The dental structure of claim 6, wherein the pigment is chosen from TiO₂, ZnO, ZnO₂, BaSO₄, talc, rice powder and combinations thereof.

14. The dental structure of claim 6, wherein the pigment is TiO₂.

15. A method for generating contrast for oral scanning on a dental structure comprising:
providing an oral coating composition, and
coating a dental structure with the oral coating composition;
wherein the oral coating composition comprises:
an acidic copolymer comprising acidic acrylate monomeric units, acidic methacrylate monomeric units, or a combination thereof;
a solvent comprising ethanol and water; and
a pigment; and
wherein the oral coating composition comprises two phases.

16. A method for scanning a dental structure comprising:
providing an oral coating composition,
coating a dental structure with the oral coating composition; and
scanning the dental structure with the oral coating composition
wherein the oral coating composition comprises:
an acidic copolymer comprising acidic acrylate monomeric units, acidic methacrylate monomeric units, or a combination thereof;
a solvent comprising ethanol and water; and
a pigment; and
wherein the oral coating composition comprises two phases.

17. The method of claim 16, further comprising removing the coating composition from the dental structure after scanning the dental structure with the oral coating composition.

18. The method of claim 17, wherein removing the coating composition from the dental structure after the scanning comprises applying an alkaline solution to the dental structure.

19. The method of claim 18, wherein removing the coating composition from the dental structure after the scanning further comprises brushing the dental structure after applying the alkaline solution to the dental structure.

20. The coating composition of claim 1, wherein the molecular weight of the acidic copolymer is from about 5,000 to about 500,000.

21. The coating composition of claim 1, wherein the acidic copolymer is chosen from Eudragit S100, Eudragit L100, Eudragit L100-55, AC210 and combinations thereof.

22. The coating composition of claim 1, wherein the pigment is chosen from cadmium orange, chrome orange, aluminum pigments, iron pigments, copper pigments, titanium pigments, tin pigments, clay earth pigments, carbon pigments, barium pigments, zinc pigments and combinations thereof.

23. The coating composition of claim 1, wherein the pigment is chosen from TiO₂, ZnO, ZnO₂, BaSO₄, talc, rice powder and combinations thereof.

24. The coating composition of claim 1, wherein the pigment is TiO₂.
